(19) United States
(12) Patent Application Publication
(10) Pub. No.: US 2009/0318749 A1
(43) Pub. Date: Dec. 24, 2009

(54) METHOD AND APPARATUS FOR PACING AND INTERMITTENT ISCHEMIA

(76) Inventors: Craig Stolen, New Brighton, MN (US); Allan C. Shuros, St. Paul, MN (US); Shantha Arcot-Krishnamurthy, Vadnais Heights, MN (US); Eric A. Mokelke, White Bear Lake, MN (US)

Correspondence Address: SCHWEGMAN, LUNDBERG & WOESSNER/BSC-CRM, PO BOX 2938, MINNEAPOLIS, MN, 55402, US

(21) Appl. No.: 12/484,822

(22) Filed: Jun. 15, 2009

Related U.S. Application Data
(60) Provisional application No. 61/074,024, filed on Jun. 19, 2008.

(51) Int. Cl. A61M 1/12; A61N 1/365

(52) U.S. Cl. 600/17; 606/108; 607/3

(57) ABSTRACT

A system delivers multiple enhanced therapies to limit myocardial damage post-revascularization. The system includes a catheter that incorporates features for delivering cardiac protection pacing therapy (CPPT) and intermittent ischemia therapy. In one embodiment, a method for delivering cardiac protection therapies to a heart is provided. One or more catheters are provided having a balloon, at least one pacing electrode and at least one hemodynamic sensor. Cardiac protection pacing therapy (CPPT) and intermittent ischemia therapy are concurrently delivered using the one or more catheters. The pacing and ischemia are adapted to protect the heart from ischemic and reperfusion injuries. The delivery of the CPPT and the intermittent ischemia are controlled using a closed-loop system monitoring a signal sensed by the at least one hemodynamic sensor.

### CROSS REFERENCE TO RELATED APPLICATIONS

This application claims the benefit of U.S. Provisional Application No. 61/074,024, filed on Jun. 19, 2008, under 35 U.S.C. § 119(e), which is hereby incorporated by reference.

This application is related to commonly assigned U.S. Patent Application Ser. No. 61/074,032, entitled “PACING CATHETER WITH EXPANDABLE DISTAL END”, filed on Jun. 19, 2008, U.S. Patent Application Ser. No. 61/074,035, entitled “PACING CATHETER FOR ACCESS TO MULTIPLE VESSELS”, filed on Jun. 19, 2008, U.S. Patent Application Ser. No. 61/074,042, entitled “PACING CATHETER RELEASING CONDUCTIVE LIQUID”, filed on Jun. 19, 2008, U.S. Patent Application Ser. No. 61/074,048, entitled “PACEMAKER INTEGRATED WITH VASCULAR INTERVENTION CATHETER”, filed on Jun. 19, 2008, U.S. Patent Application Ser. No. 61/074,055, entitled “TRANSVASCULAR BALLOON CATHETER WITH PACING ELECTRODES ON SHAFT”, filed on Jun. 19, 2008, U.S. Patent Application Ser. No. 61/074,060, entitled “PACING CATHETER WITH STENT ELECTRODE”, filed on Jun. 19, 2008, U.S. Patent Application Ser. No. 61/074,064, entitled “VASCULAR INTERVENTION CATHETERS WITH PACING ELECTRODES”, filed on Jun. 19, 2008, which are hereby incorporated by reference in their entirety.

### TECHNICAL FIELD

This application relates generally to medical devices and, more particularly, to systems, devices and methods for delivering multiple enhanced therapies, including pacing and intermittent ischemia.

### BACKGROUND

The heart is the center of a person's circulatory system. It includes an electro-mechanical system performing two major pumping functions. The left portions of the heart draw oxygenated blood from the lungs and pump it to the organs of the body to provide the organs with their metabolic needs for oxygen. The right portions of the heart draw deoxygenated blood from the body organs and pump it to the lungs where the blood gets oxygenated. These pumping functions are resulted from contractions of the myocardium. In a normal heart, the sinoatrial node, the heart's natural pacemaker, generates electrical impulses that propagate through an electrical conduction system to various regions of the heart to excite the myocardial tissues of these regions. Coordinated delays in the propagations of the electrical impulses in a normal electrical conduction system cause the various portions of the heart to contract in synchrony to result in efficient pumping functions. A blocked or otherwise abnormal electrical conduction and/or deteriorated myocardial tissue cause dysynchronous contraction of the heart, resulting in poor hemodynamic performance, including a diminished blood supply to the heart and the rest of the body. The condition where the heart fails to pump enough blood to meet the body's metabolic needs is known as heart failure.

Myocardial infarction (MI) is the necrosis of portions of the myocardial tissue resulted from cardiac ischemia, a condition in which the myocardium is deprived of adequate oxygen and metabolite removal due to an interruption in blood supply caused by an occlusion of a blood vessel such as a coronary artery. The necrotic tissue, known as infarcted tissue, loses the contractile properties of the normal, healthy myocardial tissue. Consequently, the overall contractility of the myocardium is weakened, resulting in an impaired hemodynamic performance. Following an MI, cardiac remodeling starts with expansion of the region of infarcted tissue and progresses to a chronic, global expansion in the size and change in the shape of the entire left ventricle. The consequences include a further impaired hemodynamic performance and a significantly increased risk of developing heart failure, as well as a risk of suffering recurrent MI.

Following an MI, revascularization procedures, such as percutaneous transluminal coronary angioplasty (PTCA), can be performed to reopen the occluded vessel and limit damage. While successful at lowering mortality, myocardial damage still occurs and may even be induced by the revascularization process itself, such as by cytokine release, leukocyte accumulation (neutrophil migration and activation), oxidative stress, calcium overload, side branch occlusion or distal embolism.

What is needed is a way to limit myocardial damage post-revascularization by delivering multiple enhanced therapies in a single system.

### SUMMARY

A system delivers multiple enhanced therapies to limit myocardial damage post-revascularization. The system includes a catheter that incorporates features for delivering cardiac protection pacing therapy (CPPT) and intermittent ischemia therapy.

In one embodiment, a system for delivering cardiac protection therapies to a heart via a blood vessel is provided. The system includes a catheter having at least one balloon along its length. The balloon is adapted to be placed in the blood vessel to at least partially occlude the blood vessel. The system also includes at least one pacing electrode along the length of the catheter, and at least one hemodynamic sensor near a tip of the catheter. A controller is connected to the catheter and adapted to sense a hemodynamic parameter being a measure of hemodynamic performance using the hemodynamic sensor. The controller is also adapted to control inflation and deflation of the balloon to provide intermittent ischemia, and further adapted to control pulses to the at least one pacing electrode to provide cardiac protection pacing therapy (CPPT).

In one embodiment, a guide wire catheter system for delivering cardiac protection therapies to a heart via a blood vessel is provided. The system includes a catheter having a balloon along its length. The balloon is adapted to be placed in the blood vessel to at least partially occlude the blood vessel. The system also includes a guide wire adapted to guide placement of the catheter. The guide wire includes at least one hemodynamic sensor. The system further includes at least one pacing electrode along the length of one of the catheter and the guide wire. A controller is connected to the catheter and adapted to sense a hemodynamic parameter being a measure of hemodynamic performance using the hemodynamic sensor. The controller is also adapted to control inflation and deflation of the balloon to provide intermittent ischemia, and further adapted to control pulses to the at least one pacing electrode to provide cardiac protection pacing therapy (CPPT).

In one embodiment, a method for delivering cardiac protection therapies to a heart is provided. One or more catheters are provided having a balloon, at least one pacing electrode and at least one hemodynamic sensor. Cardiac protection pacing therapy (CPPT) and intermittent ischemia therapy are concurrently delivered using the one or more catheters. The pacing and ischemia are adapted to protect the heart from ischemic and reperfusion injuries. The delivery of the CPPT and the intermittent ischemia are controlled using a closed-loop system monitoring a signal sensed by the at least one hemodynamic sensor.

This summary is an overview of some of the teachings of the present application and not intended to be an exclusive or exhaustive treatment of the present subject matter. Further details about the present subject matter are found in the detailed description and appended claims. Other aspects will be apparent to persons skilled in the art upon reading and understanding the following detailed description and viewing the drawings that form a part thereof, each of which are not to be taken in a limiting sense. The scope of the present invention is defined by the appended claims and their equivalents.

### BRIEF DESCRIPTION OF THE DRAWINGS

FIG. 1A illustrates the autonomic response to a period of exercise.

FIG. 1B illustrates the autonomic response to a period of cardiac protective pacing therapy (CPPT).

FIG. 2 is a flow chart illustrating an embodiment of a method for delivering cardiac protection therapies to a heart.

FIG. 3A is a block diagram illustrating an embodiment of a system for delivering cardiac protection therapies to a heart via a blood vessel.

FIG. 3B is a block diagram illustrating an embodiment of a guide wire catheter system for delivering cardiac protection therapies to a heart via a blood vessel.

FIG. 4 is an illustration of an embodiment of a guide catheter with pacing electrodes.

FIG. 5 is an illustration of an embodiment of a guide wire with pacing electrodes.

FIG. 6 is an illustration of an embodiment of an angioplasty catheter with pacing electrodes.

FIG. 7A is an illustration of an embodiment having electrodes incorporated into a spiral occlusion balloon.

FIG. 7B is an illustration of an embodiment having electrodes incorporated into an asymmetrical balloon catheter.

FIG. 7C is an illustration of an embodiment including a positive flow occlusion catheter.

FIG. 8A is an illustration of an embodiment of a system for delivering cardiac protection therapies to a heart via a blood vessel, and portions of an environment in which the system is used.

FIG. 8B is a block diagram illustrating an embodiment of a pacemaker providing for pacing during revascularization.

FIG. 9A is a timing diagram illustrating an embodiment of a cardioprotective pacing and alternating intermittent ischemia protocol.

FIG. 9B is a timing diagram illustrating an embodiment of a cardioprotective pacing and simultaneous intermittent ischemia protocol.

### DETAILED DESCRIPTION

The following detailed description of the present subject matter refers to the accompanying drawings which show, by way of illustration, specific aspects and embodiments in which the present subject matter may be practiced. These embodiments are described in sufficient detail to enable those skilled in the art to practice the present subject matter. Other embodiments may be utilized and structural, logical, and electrical changes may be made without departing from the scope of the present subject matter. References to “an”, “one”, or “various” embodiments in this disclosure are not necessarily to the same embodiment, and such references contemplate more than one embodiment. The following detailed description is, therefore, not to be taken in a limiting sense, and the scope is defined only by the appended claims, along with the full scope of legal equivalents to which such claims are entitled.

The present subject matter delivers cardiac protective pacing therapy (CPPT) and intermittent ischemia therapy to protect the heart from injuries. Cardiac protection pacing therapy (CPPT), also referred to as intermittent pacing therapy, has been proposed to deliver intermittent stress as a potential therapy for cardiac disease. Short bursts (10 cycles of 30 seconds on/off) of ventricular pacing during early reperfusion at physiological heart rates have been demonstrated to limit the size of cardiac injury resulting from infarction and reperfusion.

Intermittent ischemia, also referred to as vessel occlusion therapy, is another therapy that, when applied during or shortly after reperfusion, can protect the myocardium from injures associated with ischemic events, including MI. Brief periods of repetitive coronary occlusion, applied at the onset of reperfusion (post-conditioning), has been shown to attenuate myocardial injury.

The present subject matter delivers CPPT and intermittent ischemia therapy to post-MI and heart failure patients to protect the heart from ischemic and reperfusion injuries in a closed loop system using a sensed hemodynamic parameter. Embodiments include balloon catheter assemblies designed to deliver both CPPT and intermittent ischemia therapy concurrently, either during or shortly after revascularization procedures.

In one embodiment, a method for delivering cardiac protection therapies to a heart is provided. One or more catheters are provided having a balloon, at least one pacing electrode and at least one hemodynamic sensor. Cardiac protection pacing therapy (CPPT) and intermittent ischemia therapy are concurrently delivered using the one or more catheters. The pacing and ischemia are adapted to protect the heart from ischemic and reperfusion injuries. The delivery of the CPPT and the intermittent ischemia are controlled using a closed-loop system monitoring a signal sensed by the at least one hemodynamic sensor. In one embodiment, the cardiac protection pacing sequence is applied simultaneously with balloon inflation. In another embodiment, the pacing sequences alternate with, or are appended to the beginning and/or end of the balloon inflation periods.

In one embodiment the electrodes are incorporated into a spiral occlusion balloon. The spiral shape enables vessel wall contact of the pacing electrodes during periods of reperfusion while maintaining blood flow. In another embodiment, reperfusion is maintained through the use of an asymmetrically shaped balloon that contacts the vessel wall during inflation while allowing blood to perfuse through it. In another embodiment, perfusion is maintained through the use of a positive flow balloon catheter that has blood flow channels that can be opened and closed with multi lumen balloons. In one embodiment, a flow sensor or pressure sensor is added to the tip of the catheter to provide the ability to measure reperfusion. The system allows for cardiac protection pacing during controlled, gradual reperfusion using a closed loop system.

### CPPT

Autonomic tone may be modulated by stimulating or inhibiting an autonomic neural target. Embodiments of the present subject matter modulate autonomic tone using CPPT. Physiology associated with CPPT is discussed below.

The sinoatrial (SA) node generates electrical impulses that propagate through an electrical conduction system to various regions of the heart to excite the myocardial tissues of these regions. An intrinsic heart rhythm may be a normal rhythm or an abnormal rhythm. Coordinated delays in the propagations of the electrical impulses in a normal electrical conduction system cause the various portions of the heart to contract in synchrony. Synchrony, as used herein, indicates a coordinated contraction of the various portions of the heart to result in efficient pumping functions. Synchrony does not indicate that all of the portions of the heart contract at the same time.

Abnormal electrical conduction and/or deteriorated myocardial tissue cause asynchrony (no coordinated timing) between the various portions of the heart, which result in inefficient pumping functions. The present subject matter uses cardiac protective pacing therapy (CPPT) to provide a cardiac conditioning therapy to improve autonomic balance, and thus improve the health of the heart. CPPT is an intermittent pacing therapy that paces the heart in such a manner as to intentionally stress the heart during intermittent periods. When the heart is stressed with CPPT, the heart is paced, forcing the heart to work harder in comparison to a time when CPPT is not applied to the heart. The paced heart works harder in local regions of the heart away from a site where the stress-inducing pacing pulses are delivered. For example, a stressed heart may be paced to beat faster and/or more asynchronous (less coordinated). By way of example and not limitation, various CPPT embodiments increase the pacing rate for the right atrium, increase the pacing rate for the right ventricle, shorten an AV delay, and/or lengthen the VV delay. Increasing the intensity of the CPPT may involve further increasing the pacing rate of the right atrium or right ventricle, further shortening the AV delay to be more different from the intrinsic rate without CPPT, altering the pacing site, and/or further lengthening of the VV delay to be more different from the intrinsic rate without CPPT. In patients who have dysynchrony and receive biventricular pacing for the dysynchrony, cardiac stress can be increased by discontinuing the biventricular pacing during the sequence of stress inducing pacing pulses. Decreasing the intensity of the CPPT may involve altering the pacing site, may involve reducing the pacing rate of the right atrium or right ventricle closer to the intrinsic rate, may involve increasing the AV delay closer to the intrinsic AV delay, and/or may involve shortening the VV delay closer to the intrinsic VV delay (whether or not the intrinsic rhythm is normal or abnormal). Delivering CPPT with higher intensity (not stress) corresponds to increasing the sympathetic response during the CPPT.

### Diseases

The present subject matter can be used to prophylactically or therapeutically treat various diseases by modulating autonomic tone. Examples of such diseases or conditions include hypertension, cardiac remodeling, and heart failure.

Hypertension is a cause of heart disease and other related cardiac co-morbidities. Hypertension occurs when blood vessels constrict. As a result, the heart works harder to maintain flow at a higher blood pressure, which can contribute to heart failure. Hypertension generally relates to high blood pressure, such as a transitory or sustained elevation of systemic arterial blood pressure to a level that is likely to induce cardiovascular damage or other adverse consequences. Hypertension has been defined as a systolic blood pressure above 140 mm Hg or a diastolic blood pressure above 90 mm Hg. Consequences of uncontrolled hypertension include, but are not limited to, retinal vascular disease and stroke, left ventricular hypertrophy and failure, myocardial infarction, dissecting aneurysm, and renovascular disease. A large segment of the general population, as well as a large segment of patients implanted with pacemakers or defibrillators, suffer from hypertension. The long term mortality as well as the quality of life can be improved for this population if blood pressure and hypertension can be reduced. Many patients who suffer from hypertension do not respond to treatment, such as treatments related to lifestyle changes and hypertension drugs.

Following myocardial infarction (MI) or other cause of decreased cardiac output, a complex remodeling process of the ventricles occurs that involves structural, biochemical, neurohormonal, and electrophysiologic factors. Ventricular remodeling is triggered by a physiological compensatory mechanism that acts to increase cardiac output due to so-called backward failure which increases the diastolic filling pressure of the ventricles and thereby increases the so-called preload (i.e., the degree to which the ventricles are stretched by the volume of blood in the ventricles at the end of diastole). An increase in preload causes an increase in stroke volume during systole, a phenomena known as the Frank-Starling principle. When the ventricles are stretched due to the increased preload over a period of time, however, the ventricles become dilated. The enlargement of the ventricular volume causes increased ventricular wall stress at a given systolic pressure. Along with the increased pressure-volume work done by the ventricle, this acts as a stimulus for hypertrophy of the ventricular myocardium. The disadvantage of dilatation is the extra workload imposed on normal, residual myocardium and the increase in wall tension (Laplace's Law) which represent the stimulus for hypertrophy. If hypertrophy is not adequate to match increased tension, a vicious cycle ensues which causes further and progressive dilatation. As the heart begins to dilate, afferent baroreceptor and cardiopulmonary receptor signals are sent to the vasomotor central nervous system control center, which responds with hormonal secretion and sympathetic discharge. It is the combination of hemodynamic, sympathetic nervous system and hormonal alterations (such as presence or absence of angiotensin converting enzyme (ACE) activity) that ultimately account for the deleterious alterations in cell structure involved in ventricular remodeling. The sustained stresses causing hypertrophy induce apoptosis (i.e., programmed cell death) of cardiac muscle cells and eventual wall thinning which causes further deterioration in cardiac function. Thus, although ventricular dilation and hypertrophy may at first be compensatory and increase cardiac output, the processes ultimately result in both systolic and diastolic dysfunction (decompensation). It has been shown that the extent of ventricular remodeling is positively correlated with increased mortality in post-MI and heart failure patients.

Heart failure refers to a clinical syndrome in which cardiac function causes a below normal cardiac output that can fall below a level adequate to meet the metabolic demand of peripheral tissues. Heart failure may present itself as congestive heart failure (CHF) due to the accompanying venous and pulmonary congestion. Heart failure can be due to a variety of etiologies such as ischemic heart disease. Heart failure patients have reduced autonomic balance, which is associated with LV dysfunction and increased mortality. Modulation of the sympathetic and parasympathetic nervous systems has potential clinical benefit in preventing remodeling and death in heart failure and post-MI patients. Direct electrical stimulation can activate the baroreflex, inducing a reduction of sympathetic nerve activity and reducing blood pressure by decreasing vascular resistance. Sympathetic inhibition and parasympathetic activation have been associated with reduced arrhythmia vulnerability following a myocardial infarction, presumably by increasing collateral perfusion of the acutely ischemic myocardium and decreasing myocardial damage.

### Therapy Protocols

The present subject matter modulates autonomic tone using CPPT and intermittent ischemia therapy. Preconditioning of the myocardium occurs as a prophylactic therapy in preparation for an anticipated event. For example, the myocardium can be preconditioned in anticipation for surgery, or can be preconditioned based on observed or detected events that indicate an increased probability of an upcoming ischemic event. Examples of such events include a previous myocardial infarction and angina. Prophylactic conditioning can be used to modulate autonomic tone from higher sympathetic tendencies toward an autonomic balance to improve the health of a patient prone to heart failure, hypertension and remodeling. Postconditioning of the myocardium occurs as a therapeutic treatment to a disease. For example, postconditioning of the myocardium can reduce the size of any infarct area caused by the ischemic event. For example, the postconditioning therapy can be triggered based on commands received from a patient or physician after observing a myocardial infarction, or a physician can deliver postconditioning therapy after a surgical procedure for which the heart was stopped. In an embodiment, the device detects an ischemic event or other event indicated for postconditioning therapy, and automatically delivers the postconditioning therapy. The postconditioning therapy can occur during the time of reperfusion, for a time after reperfusion, or during and for a time after reperfusion.

Intermittent ischemia, also referred to as vessel occlusion therapy, is another therapy that, when applied during or shortly after reperfusion, can protect the myocardium from injures associated with ischemic events, including MI. Brief periods of repetitive coronary occlusion, applied at the onset of reperfusion (post-conditioning), has been shown to attenuate myocardial injury. Combining intermittent ischemia therapy with CPPT, as proposed herein, can better protect the heart from ischemic and reperfusion injuries.

A cardiac conditioning therapy may also be referred to as a cardiac protective therapy, as it is protects against the deleterious effects of an autonomic tone with an undesirably high sympathetic tendency. The cardiac conditioning therapy may mimic the effects of exercise.

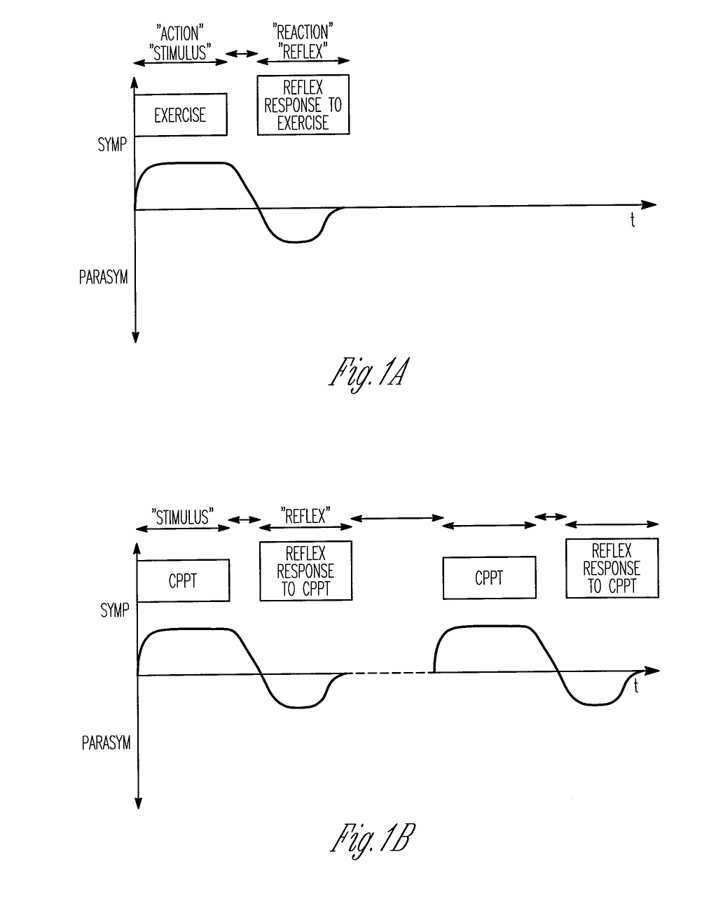

FIG. 1A illustrates the autonomic response to a period of exercise. Exercise is a stimulus that increases the sympathetic response. After the period of exercise ends, a reflex response to the exercise increases the parasympathetic tone. The parasympathetic response appears to be a reaction to the sympathetic action of exercise. Those of ordinary skill in the art will understand that the illustrated graph is a simple illustration. The horizontal axis represents time, and the vertical axis represents the autonomic tone. For simplicity, the value of the vertical axis corresponding to the horizontal axis represents the autonomic balance (the balance between the sympathetic and parasympathetic neural activity). Those of ordinary skill in the art will know that, over time, as the health of the heart improves and the autonomic balance improves by having a more parasympathetic tone, the horizontal axis (representing the autonomic balance) will trend more toward the parasympathetic tone. By way of an everyday example of exercise, it is noted that a runner's resting heart rate tends to lower as the runner's conditioning improves. This example indicates that running, which temporarily increases sympathetic tone as evidenced by an increased heart rate, will trend the autonomic balance of the runner toward a more parasympathetic tone.

FIG. 1B illustrates the autonomic response to a period of CPPT. Similar to the period of exercise, CPPT is a stimulus that increases the sympathetic response during the period of pacing, and results in a reflex response that increases parasympathetic tone after the pacing ends. As illustrated, the CPPT functions as a stimulus that provides a sympathetic component (action) that generates a desired parasympathetic reflex (reaction to the action). A cardiac conditioning therapy may correspond to recommended exercises periods (e.g. 30 to 60 minutes, two times per day). Various therapy durations and frequencies can be used. Various cardiac conditioning therapies are programmed according to a schedule. Various cardiac conditioning therapies are programmed to occur after a detected event such as a period of exercise by the patient.

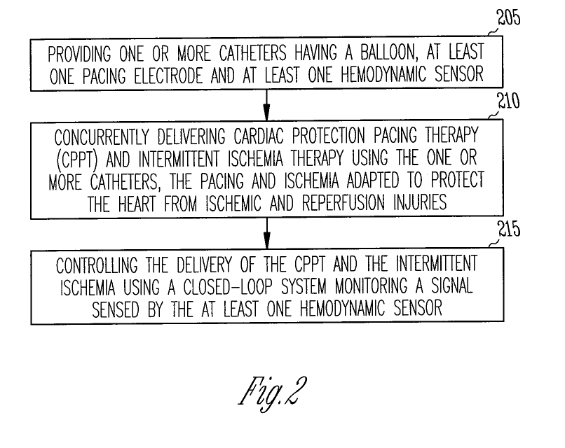

FIG. 2 is a flow chart illustrating an embodiment of a method for delivering cardiac protection therapies to a heart. At 205, one or more catheters are provided having a balloon, at least one pacing electrode and at least one hemodynamic sensor. Cardiac protection pacing therapy (CPPT) and intermittent ischemia therapy are concurrently delivered at 210 using the one or more catheters. The pacing and ischemia are adapted to protect the heart from ischemic and reperfusion injuries. At 215, the delivery of the CPPT and the intermittent ischemia are controlled using a closed-loop system monitoring a signal sensed by the at least one hemodynamic sensor.

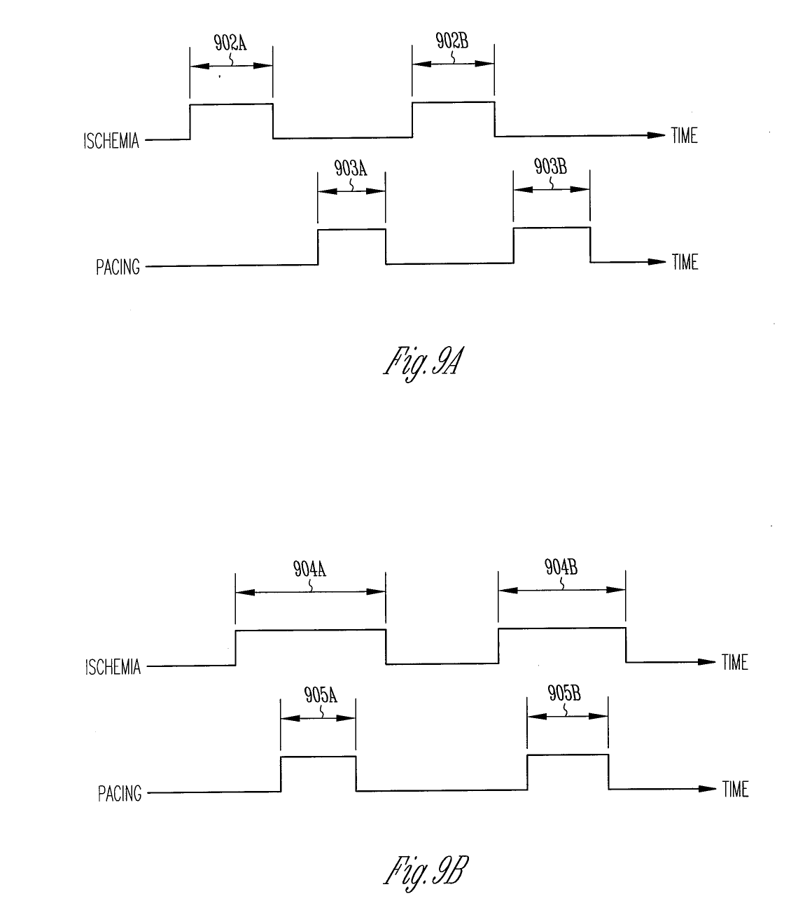

According to one embodiment, CPPT and intermittent ischemia are delivered alternately, as depicted in FIG. 9A. CPPT is delivered when the balloon is deflated, for example. According to another embodiment, CPPT and intermittent ischemia are delivered simultaneously, as depicted in FIG. 9B. Delivering intermittent ischemia therapy includes inflating and deflating the balloon, and balloon inflation is gated to an electrocardiogram, such as based on a number of R-waves in the electrocardiogram in an embodiment. In one example, a spiral balloon is used and adapted to enable vessel wall contact of the pacing electrode during periods of reperfusion while maintaining blood flow. An asymmetrically shaped balloon is used in another example, the balloon contacting the vessel wall during inflation to maintain reliable contact between the electrode and/or sensor and the vessel wall. According to an embodiment, the one or more catheters have at least one flow sensor. The one or more catheters have at least one pressure sensor, in an embodiment.

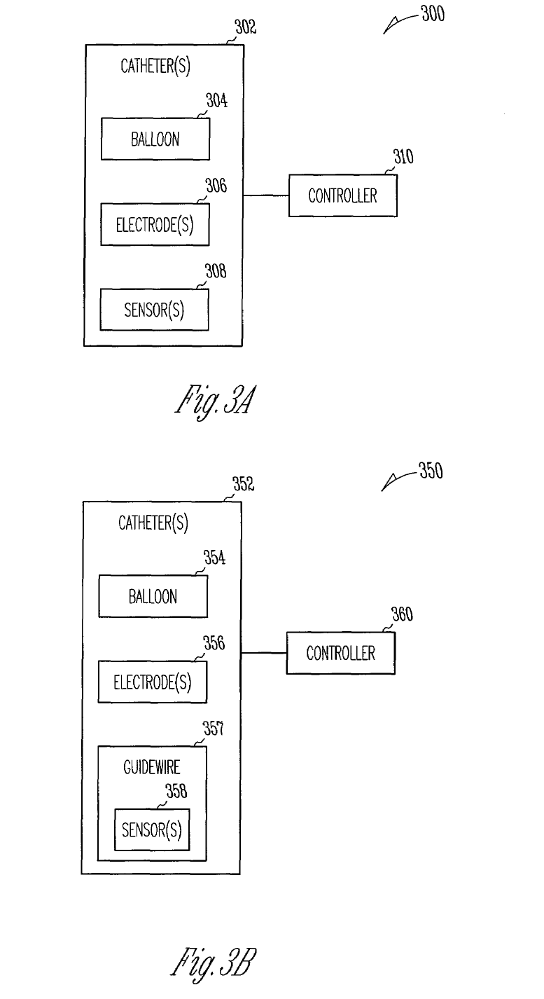

FIG. 3A is a block diagram illustrating an embodiment of a system for delivering cardiac protection therapies to a heart via a blood vessel. The system 300 includes at least one catheter 302 having at least one balloon 304 along its length. In an embodiment, the catheter 302 includes a transcutaneous transluminal catheter. The balloon 304 is adapted to be placed in the blood vessel to at least partially occlude the blood vessel. The system 300 also includes at least one pacing electrode 306 along the length of the at least one catheter, and at least one hemodynamic sensor 308 near a tip of the catheter. A controller 310 is connected to the at least one catheter 302 and adapted to sense a hemodynamic parameter being a measure of hemodynamic performance using the hemodynamic sensor 308. The controller 310 is also adapted to control inflation and deflation of the balloon 304 to provide intermittent ischemia, and further adapted to control pulses to the at least one pacing electrode 306 to provide cardiac protection pacing therapy (CPPT). The intermittent ischemia therapy and the CPPT are adapted to protect the heart from ischemic and reperfusion injuries in a closed loop system using the sensed hemodynamic parameter, in various embodiments.

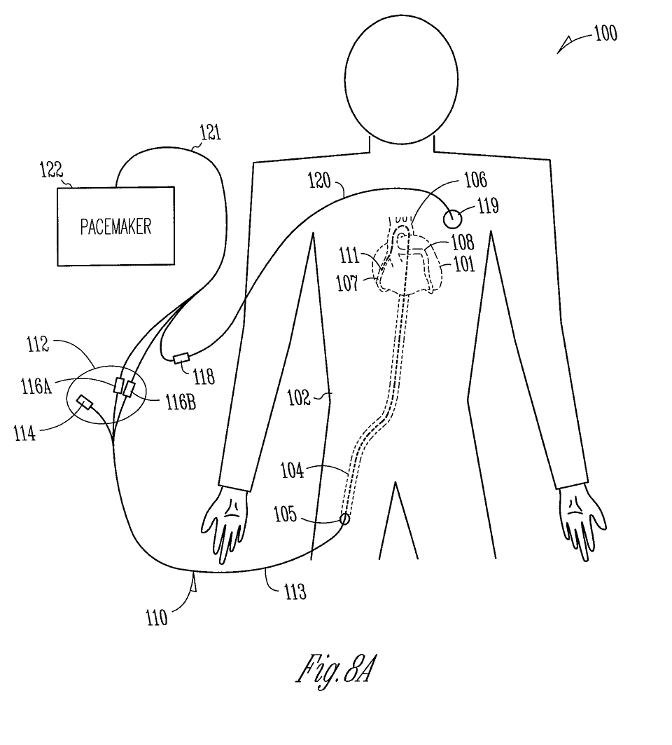

According to an embodiment, the at least one balloon includes a spiral balloon, such as the balloon in FIG. 7A, adapted to enable vessel wall contact of the pacing electrode during periods of reperfusion while maintaining blood flow. Two balloons are used assist in delivering cardiac protection therapies, in an embodiment. The two balloons include an outer balloon facing the vessel wall and adapted to apply pressure against the vessel wall without filling an inner lumen of the spiral, and an inner balloon facing a vessel lumen and adapted to fill to occlude blood flow. The outer balloon can includes at least one electrode. In various embodiments, the balloon includes an asymmetrically shaped balloon that contacts the vessel wall during inflation while still allowing blood to perfuse through the balloon. The catheter includes a positive flow balloon catheter that includes one or more blood flow channels that can be opened and closed using multiple lumen balloons, such as in the embodiment shown in FIG. 7B. The at least one hemodynamic sensor can include a flow sensor, a pressure sensor, an ultrasound sensor, or other sensor for sensing a property of blood. An embodiment includes at least one sensing electrode along the length of the catheter. In various embodiments, the at least one catheter is adapted to deploy a stent.

FIG. 3B is a block diagram illustrating an embodiment of a guide wire catheter system for delivering cardiac protection therapies to a heart via a blood vessel. The system 350 includes at least one catheter 352 having a balloon 354 along its length. The balloon 354 is adapted to be placed in the blood vessel to at least partially occlude the blood vessel. The system 350 also includes at least one pacing electrode 356 along the length of the at least one catheter, and a guide wire 357 adapted to guide placement of the catheter. The at least one catheter is transluminally advanced over the guide wire, according to various embodiments. The electrode 356 can be on the guide wire 357, in an embodiment. The guide wire 357 includes at least one hemodynamic sensor 358. A controller 360 is connected to the at least one catheter 352 and adapted to sense a hemodynamic parameter being a measure of hemodynamic performance using the hemodynamic sensor 358. The controller 360 is also adapted to control inflation and deflation of the balloon 354 to provide intermittent ischemia, and further adapted to control pulses to the at least one pacing electrode 356 to provide cardiac protection pacing therapy (CPPT). The intermittent ischemia therapy and the CPPT are adapted to protect the heart from ischemic and reperfusion injuries in a closed loop system using the sensed hemodynamic parameter, in various embodiments. According to an embodiment, the guide wire including the at least one hemodynamic sensor includes a flow sensor, sometimes referred to as flow wire. According to another embodiment, the guide wire including the at least one hemodynamic sensor includes a pressure sensor, sometimes referred to as pressure wire. An embodiment includes at least one sensing electrode along the length of at least one of the catheter and the guide wire.

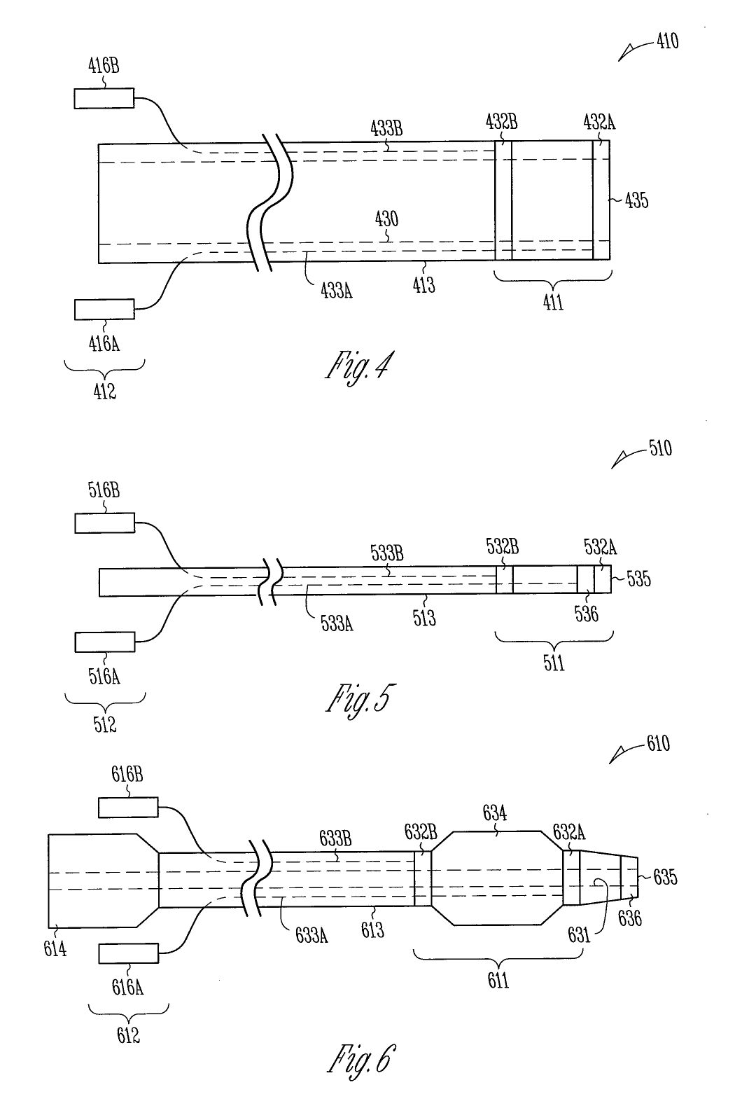

FIGS. 4-6 illustrate a percutaneous transluminal vascular intervention (PTVI) device assembly that includes a guide catheter, a guide wire, and an angioplasty catheter. When a blood vessel such as the coronary artery is partially or completely occluded, a revascularization procedure such as percutaneous transluminal coronary angioplasty (PTCA) can be performed to reopen the occluded blood vessel. During a revascularization procedure such as a PTCA procedure, the guide catheter is inserted into the patient first, followed by the guide wire through a lumen of the guide catheter. The angioplasty catheter includes a lumen that accommodates a portion of the guide wire, thereby allowing the angioplasty catheter to be inserted into the patient through the guide catheter and over the guide wire. The guide catheter, guide wire, and angioplasty catheter are inserted in such a way that allows an angioplasty device, such as a balloon, of the angioplasty catheter to be placed in the portion of a blocked blood vessel that is to be reopened during the revascularization procedure.

FIG. 4 is an illustration of an embodiment of a guide catheter 410. Guide catheter 410 is an embodiment of PTVI device 110 and has an elongate shaft 413 between a distal end portion 411 and a proximal end portion 412. Distal end portion 411 is configured for intravascular placement and includes a distal tip 435. A lumen 430 extends within shaft 413 and has a proximal opening in proximal end portion 412 and a distal opening at distal tip 435. Lumen 430 accommodates at least a portion of the angioplasty catheter. Distal end portion 411 includes pacing electrodes 432A-B. In the illustrated embodiment, electrode 432A is incorporated onto distal tip 435. Conductor 433A is connected between pacing electrode 432A and a connector 416A. Conductor 433B is connected between pacing electrode 432B and a connector 416B. Connectors 416A-B are each part of proximal end portion 412. In one embodiment, conductors 433A-B each extend longitudinally within shaft 413. In another embodiment, conductors 433A-B each extend longitudinally on the outer surface of shaft 413 and are insulated.

In one embodiment, guide catheter 410 has a length in a range of approximately 50 cm to 150 cm. Shaft 413 has an outer diameter in a range of approximately 0.5 mm to 8 mm, and lumen 430 has a diameter in a range of approximately 0.4 mm to 7 mm. Conductors 433A-B are made of a metallic material such as stainless steel or an alloy of nickel, titanium, and/or cobalt. Elongate shaft 413 is made of a material such as silicone, polyurethane, Teflon, or polytetrafluoroethylene (PTFE). Electrodes 432A-B are made of a metallic material such as platinum or an iridium alloy.

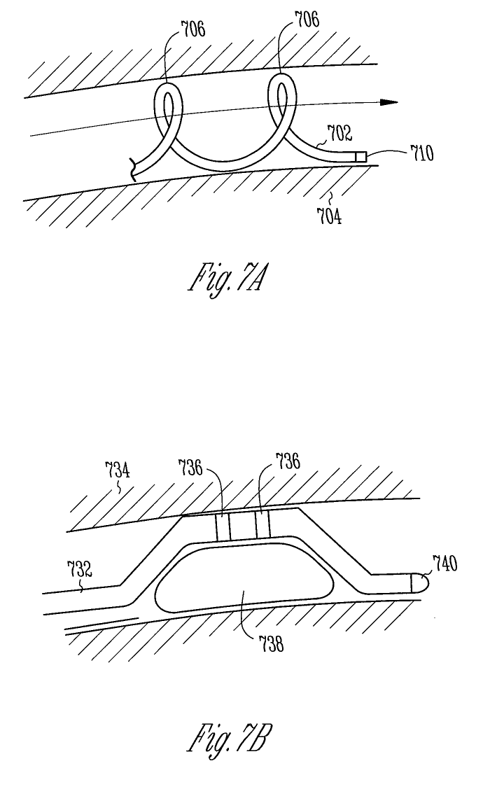

FIG. 5 is an illustration of an embodiment of a guide wire 510. Guide wire 510 is an embodiment of PTVI device 110 and has an elongate shaft 513 between a distal end portion 511 and a proximal end portion 512. Distal end portion 511 is configured for intravascular placement and includes a distal tip 535. Distal end portion 511 includes pacing electrodes 532A-B. In the illustrated embodiment, electrode 532A is incorporated onto distal tip 535. Conductor 533A is connected between pacing electrode 532A and a connector 516A. Conductor 533B is connected between pacing electrode 532B and a connector 516B. Connectors 516A-B are each part of proximal end portion 512. In one embodiment, conductors 533A-B each extend longitudinally within shaft 513. In another embodiment, conductors 533A-B each extend longitudinally on the outer surface of shaft 513 and are insulated. In one embodiment, one of connectors 533A-B is the core of guide wire 510. According to an embodiment, a sensor 536 is located near distal tip 535. More than one sensor may be used, in an embodiment.

In one embodiment, guide wire 510 has a length in a range of approximately 30 cm to 300 cm. Shaft 513 is an elongate cylindrical shaft having a diameter in a range of approximately 0.2 mm to 1.5 mm. Conductors 533A-B are made of a metallic material such as stainless steel or an alloy of nickel, titanium, and/or cobalt. Elongate shaft 513 is made of a material such as silicone, polyurethane, Teflon, or polytetrafluoroethylene (PTFE). Electrodes 532A-B are made of a metallic material such as platinum or an iridium alloy.

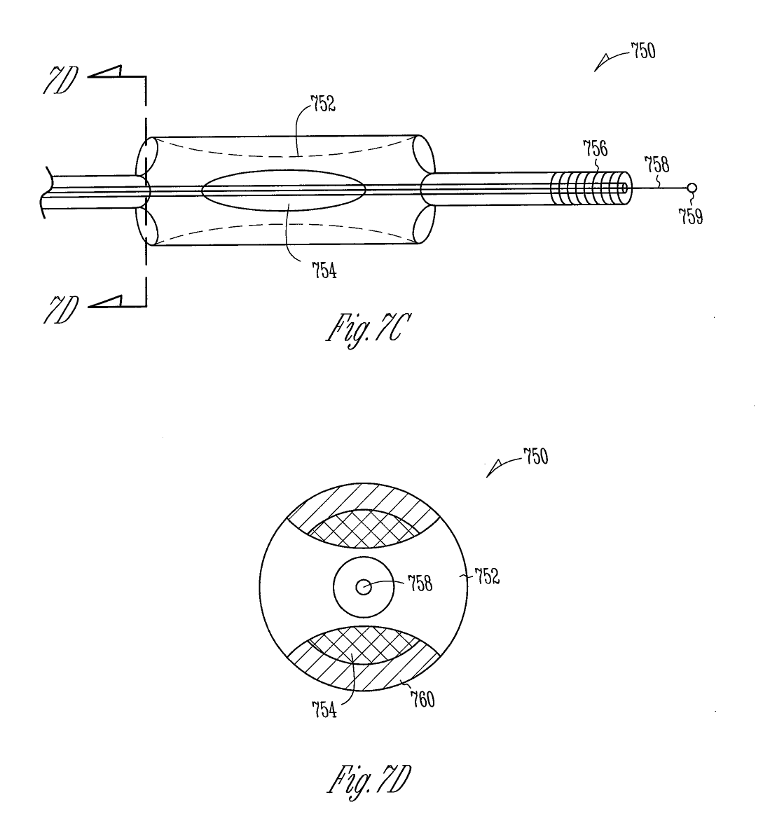

FIG. 6 is an illustration of an embodiment of an angioplasty catheter 610. Angioplasty catheter 610 is an embodiment of PTVI device 110 and has an elongate shaft 613 between a distal end portion 611 and a proximal end portion 612. A lumen 631 longitudinally extends within shaft 613 to accommodate at least a portion of a guide wire such as guide wire 510. Distal end portion 611 is configured for intravascular placement and includes a distal tip 635 and an angioplasty device 634. Angioplasty device 634 has one end approximately adjacent to distal tip 635 and another end coupled to shaft 613. In one embodiment, angioplasty device 634 includes an adjustable portion that has controllable expandability and contractibility. In the illustrated embodiment, angioplasty device 634 includes a balloon that is inflated and deflated through a lumen longitudinally extending within shaft 613 and connected between the chamber of the balloon and a connector 614 at proximal end portion 612. The balloon is inflatable using an air or liquid pump connected to that connector. In various embodiments, angioplasty device 634 includes a balloon or other device that allows for application of an angioplasty therapy such as vascular dilatation, stent delivery, brachytherapy (radiotherapy), atherectomy, or embolic protection. In one embodiment, distal tip 635 is a tapered tip that facilitates the insertion of angioplasty catheter 610 into a blood vessel. Distal end portion 611 includes pacing electrodes 632A-B. In the illustrated embodiment, pacing electrode 632A is approximately adjacent to one end of angioplasty device 634, and pacing electrode 632B is approximately adjacent to the other end of angioplasty device 634. A conductor 633A extends longitudinally within shaft 613 and is connected between pacing electrode 632A and a pacing connector 616A, which is part of proximal end portion 612. A conductor 633B extends longitudinally within elongate shaft 613 and is connected between pacing electrode 632B and a pacing connector 616B, which is also part of proximal end portion 612. In an alternative embodiment, pacing connectors 616A-B are physically integrated into one multi-conductor connector. Proximal end portion 612 also includes a proximal end device 614. In various embodiments, connector 614 includes a structure that accommodates all the mechanical connection and access requirements for angioplasty catheter 610, which depend on the function of angioplasty device 634. In one embodiment, connector 614 includes an integrated device. In another embodiment, connector 614 branches out into multiple connectors and/or other devices. According to an embodiment, a sensor 636 is located near distal tip 635. More than one sensor may be used, in an embodiment.

In one embodiment, angioplasty catheter 610 has a length in a range of approximately 50 cm to 150 cm. Shaft 613 is an elongate cylindrical shaft having a diameter in a range of approximately 1 mm to 5 mm. In one embodiment, angioplasty device 634 has an adjustable, substantially cylindrical or semi-spherical shape with a maximum diameter in a range of approximately 1 mm to 10 mm when fully expanded and a maximum diameter in a range of approximately 0.5 mm to 5 mm when fully contracted. In one embodiment, conductors 633A-B are each made of a metallic material such as stainless steel or an alloy of nickel, titanium, and/or cobalt. Electrodes 632A-B are each made of a metallic material such as platinum or an iridium alloy. Elongate shaft 613 has a tubular outer shell made of a material such as silicone, polyurethane, Teflon, or polytetrafluoroethylene (PTFE).

Guide catheter 410, guide wire 510, and angioplasty device 610 are illustrated in FIGS. 4-6 for illustrative but not restrictive purposes. For example, one or more pacing electrodes can be distributed on each of these PTVI devices in any way allowing delivery of pacing pulses to desirable locations. In various embodiments, one or more pacing electrodes are incorporated onto one or more of guide catheter 410, guide wire 510, and angioplasty device 610 for delivering pacing pulses through the PTVI device assembly including these three devices. In one embodiment, one or more defibrillation electrodes are also incorporated onto one or more of guide catheter 410, guide wire 510, and angioplasty device 610 for delivering defibrillation shocks through the PTVI device assembly. In one embodiment, one or more pacing electrodes such as one of more of pacing electrodes 432A-B, 532A-B, and 632A-B are made of conductive radiopaque material to function as one or more radiopaque markers for locating guide catheter 410, guide wire 510, and/or angioplasty device 610 using fluoroscopy.

In one embodiment, angioplasty device 610 includes a balloon. Guide wire 510 remains within lumen 631 when the balloon is inflated. The inflated balloon is over pacing electrodes 532A-B. When being deflated, the balloon is retracted to expose electrodes 532A-B, thereby allowing delivery of pacing pulses. In one embodiment, shaft 613 includes a portion having an adjustable length that is shortened to expose electrodes 532A-B when the balloon is deflated.

In one application during a PTCA procedure for reopening, for example, right coronary artery 107, guide catheter 410 is inserted into femoral artery 104 and advanced to aorta 106 until distal tip 435 reaches the point where right coronary artery 107 branches from aorta 106. Guide wire 510 is introduced through lumen 430 of guide catheter 410 until distal end 535 is in right coronary artery 107. Angioplasty catheter 610 is then introduced through lumen 430 over guide wire 510 until angioplasty device 634 (balloon) is in the portion of right coronary artery 107. In one embodiment, the acute pacing cardioprotection therapy is delivered using electrodes 432A-B as soon as guide catheter 510 is in place for the PTCA procedure. In one embodiment, when the PTVI device assembly including guide catheter 410, guide wire 510, and angioplasty device 610 are in place for the PTCA procedure, the acute pacing cardioprotection therapy is delivered using one or more pairs of pacing electrodes selected from electrodes 432A-B, 532A-B, 632A-B, and 119.

FIG. 7A is an illustration of an embodiment having electrodes incorporated into a spiral occlusion balloon. One or more spiral balloons 702 along catheter 704 have electrodes 706. The placement of the electrodes and the spiral configuration of the balloon allow vessel wall contact of the pacing electrodes during periods of occlusion and reperfusion. According to an embodiment, the spiral has separate opposed balloons. An outer balloon faces the vessel wall, and applies pressure against the vessel wall without filling the inner lumen of the spiral. Electrodes can also be incorporated into the outer balloon. The inner balloon faces the vessel lumen and fills to occlude blood flow, in an embodiment. According to an embodiment, a sensor 710 is located along the catheter. More than one sensor may be used, in an embodiment.

FIG. 7B is an illustration of an embodiment having electrodes incorporated into an asymmetrical balloon catheter. The shaft 732 of the catheter on the side of the balloon 738 is asymmetrical, in an embodiment. One or more balloons 738 along asymmetrical catheter shaft 732 have electrodes 736. The placement of the electrodes allows vessel wall contact of the pacing electrodes during periods of occlusion and reperfusion. When inflated, the balloon 738 pushes the catheter body with electrodes 736 against the vessel wall 734. According to an embodiment, a sensor 740 is located near the distal tip of the catheter. More than one sensor may be used, in an embodiment.

FIG. 7C is an illustration of an embodiment including a positive flow occlusion catheter 750. Blood flow channels can be opened and closed with multi-lumen balloons. Outer balloon 752 deploys a stent, and also pulls open a blood bypass channel 760 (see cross section 770) with inflation of the outer balloon. Inner balloon 754 is adapted to occlude the vessel for delivery of intermittent ischemia therapy. Electrode(s) 756 along the catheter 750 are adapted to provide CPPT according to a programmed schedule, in an embodiment. Guide wire 758 may incorporate one or more sensors, such as a flow or pressure wire, in various embodiments. According to an embodiment, a sensor 759 is located near the distal tip of the catheter. More than one sensor may be used, in an embodiment.

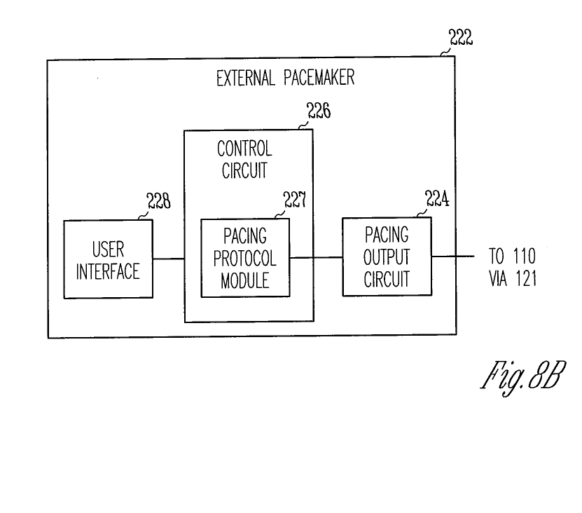

FIG. 8A is an illustration of an embodiment of a system 100 for delivering cardiac protection therapies to a heart via a blood vessel, and portions of an environment in which the system is used. System 100 includes a PTVI device 110, a pacemaker 122, and a cable 121 connecting PTVI device 110 and pacemaker 122. When needed, system 100 also includes a reference electrode 119, which is a surface electrode, such as a skin patch electrode, connected to a lead 120. Lead 120 is connected to a connector 118 allowing its connection to cable 121.

PTVI device 110 is used during a revascularization procedure and includes a distal end portion 111 for intravascular placement and a proximal end portion 112. Proximal end portion 112 includes a proximal end device 114 and pacing connectors 116A-B. Proximal end device 114 includes various connectors and other structures allowing manipulation of PTVI device 110 including the percutaneous transluminal insertion of the device and operation of an angioplasty device at distal end 111. Pacing connectors 116A-B provide for electrical connections between pacemaker 122 and PTVI device 110 through cable 121. In the illustrated embodiment, PTVI device 110 is a PTCA device used in a PTCA procedure. During the PTCA procedure, an opening 105 is made on a femoral artery 104 in a patient's body 102. PTVI device 110 is inserted into femoral artery 104 and advanced to an aorta 106 and then to a right coronary artery 107, which is narrowed or blocked. The angioplasty device at distal end 111 is then used to open up the blocked right coronary artery 107. In another embodiment, PTVI device 110 is used to open up a blocked left coronary artery 108.

Distal end portion 111 of PTVI device 110 includes one or more pacing electrodes to allow pacing pulses to be delivered to a heart 101 during the PTCA procedure. In one embodiment, pacing pulses are delivered through two pacing electrodes on distal end portion 111 of PTVI device 110. In another embodiment, pacing pulses are delivered through a pacing electrode on distal end portion 111 of PTVI device 110 and surface electrode 119 functioning as the return electrode for pacing.

Pacemaker 122 delivers pacing pulses by executing a cardioprotective pacing protocol. In one embodiment, the cardioprotective pacing protocol specifies a cardioprotective pacing sequence for preventing arrhythmias and cardiac injuries associated with the revascularization procedure. In one embodiment, pacemaker 122 is an external pacemaker such as a PSA. In another embodiment, pacemaker 122 includes an implantable pacemaker adapted for external use.

It is to be understood that FIG. 8A is for illustrative, but not restrictive, purposes. For example, the physical structure of proximal end portion 112 depends on functional and ease-of-use considerations. Proximal end device 114 represents a structure that accommodates all the mechanical connection and access requirements, which depend on the specific configuration and function of PTVI device 110. In one embodiment, proximal end device 114 includes an integrated device as illustrated in FIG. 8A. In another embodiment, proximal end device 114 branches out into multiple connectors and/or other devices. Pacing connectors 116A-B represent a structure that accommodates all the electrical connections required for delivering pacing pulses from pacemaker 122 to PTVI device 110. The number of pacing connectors depends on the number of pacing electrodes incorporated onto PTVI device 110 and how it is to be connected to cable 121. In one embodiment, when more than one electrical connection is needed for delivering the pacing pulses, proximal end portion 112 includes branched-out pacing connectors such as pacing connectors 116 and 117 as illustrated in FIG. 8A. In another embodiment, proximal end portion 112 includes a single connector providing for multiple, independent electrical connections.

FIG. 8B is a block diagram illustrating an embodiment of an external pacemaker 222 that provides for pacing during revascularization. External pacemaker 222 is an embodiment of pacemaker 122 and includes a pacing output circuit 224, a user interface 228, and a control circuit 226. Pacing output circuit 224 delivers pacing pulses to PTVI device 110 through cable 121. User interface 228 allows a user to control the delivery of the pacing pulses by controlling pacing parameters and/or timing of the delivery. Control circuit 226 controls the delivery of the pacing pulses. In one embodiment, external pacemaker 222 is a PSA including a chassis that houses pacing output circuit 224 and control circuit 226. User interface 228 is incorporated onto the chassis.

In the illustrated embodiment, control circuit 226 includes a pacing protocol module 227, which enables control circuit 226 to control the delivery of the pacing pulses by automatically executing a pacing protocol. To provide an acute pacing cardioprotection therapy, the pacing protocol specifies a cardioprotective pacing sequence that includes alternating pacing and non-pacing periods for delivering pacing during a revascularization procedure such as a PTCA procedure.

In one embodiment, pacing protocol module 227 is configured to be detachably connected to external pacemaker 222. In a specific embodiment, pacing protocol module 227 includes a memory device that stores the cardioprotective pacing protocol, and control circuit 226 is capable of automatically executing the cardioprotective pacing protocol when pacing protocol module 227 is connected to external pacemaker 222. In another specific embodiment, in addition to the memory device that stores the cardioprotective pacing protocol, pacing protocol module 227 includes a user interface that allows the user to adjust parameters of the cardioprotective pacing protocol and/or control circuitry that supplement the functions of control circuit 226 for automatically executing the cardioprotective pacing protocol. In various embodiments, other pacing protocol modules are provided for automatically executing pacing protocols using external pacemaker 222. In various embodiments, the user is provided with external pacemaker 222 and pacing protocol modules for executing pacing protocols such as the cardioprotective pacing protocol, cardiac resynchronization therapy (CRT) pacing protocol, and cardiac remodeling control therapy (RCT) pacing protocol. Compared to a PSA that requires the user to manually adjust pacing parameters during a test or therapy session, the automatic execution of the pacing protocol increases the accuracy of pacing control and reduces or eliminates the need for the user to control the delivery of the pacing pulses, so that the user can more attentive to the response of the patient and/or the revascularization procedure.

FIG. 9A is a timing diagram illustrating an embodiment of a cardioprotective pacing and alternating intermittent ischemia protocol. Time periods 902A and 902B indicate periods during which the vessel is occluded using the balloon(s). Time periods 903A and 903B indicate periods during which pulses are delivered via electrode(s) in the catheter. In this embodiment, pacing is only delivered when the vessel is not being occluded.

FIG. 9B is a timing diagram illustrating an embodiment of a cardioprotective pacing and simultaneous intermittent ischemia protocol. Time periods 904A and 904B indicate periods during which the vessel is occluded using the balloon(s). Time periods 905A and 905B indicate periods during which pulses are delivered via electrode(s) in the catheter. In this embodiment, pacing is delivered when the vessel is being occluded. Other embodiments, such as overlap of pacing and ischemia therapy, are also possible without departing from the scope of this disclosure. The timing shown in FIGS. 9A and 9B are only examples (e.g. 904A may be equal to 904B, in an embodiment).

One of ordinary skill in the art will understand that, the modules and other circuitry shown and described herein can be implemented using software, hardware, and combinations of software and hardware. As such, the terms module and circuitry, for example, are intended to encompass software implementations, hardware implementations, and software and hardware implementations.

The methods illustrated in this disclosure are not intended to be exclusive of other methods within the scope of the present subject matter. Those of ordinary skill in the art will understand, upon reading and comprehending this disclosure, other methods within the scope of the present subject matter. The above-identified embodiments, and portions of the illustrated embodiments, are not necessarily mutually exclusive. These embodiments, or portions thereof, can be combined. In various embodiments, the methods are implemented using a computer data signal embodied in a carrier wave or propagated signal, that represents a sequence of instructions which, when executed by one or more processors cause the processor(s) to perform the respective method. In various embodiments, the methods are implemented as a set of instructions contained on a computer-accessible medium capable of directing a processor to perform the respective method. In various embodiments, the medium is a magnetic medium, an electronic medium, or an optical medium.

The above detailed description is intended to be illustrative, and not restrictive. Other embodiments will be apparent to those of skill in the art upon reading and understanding the above description. The scope of the invention should, therefore, be determined with reference to the appended claims, along with the full scope of equivalents to which such claims are entitled.

What is claimed is:

1. A system for delivering cardiac protection therapies to a heart via a blood vessel, comprising:
a catheter having at least one balloon along its length, the balloon adapted to be placed in the blood vessel to at least partially occlude the blood vessel;
at least one pacing electrode along the length of the catheter;
at least one hemodynamic sensor near a tip of the catheter; and
a controller connected to the catheter and adapted to sense a hemodynamic parameter being a measure of hemodynamic performance using the hemodynamic sensor, and adapted to control inflation and deflation of the balloon to provide intermittent ischemia, and further adapted to control pulses to the at least one pacing electrode to provide cardiac protection pacing therapy (CPPT).

2. The system of claim 1, wherein the at least one balloon includes a spiral balloon, the spiral balloon adapted to enable vessel wall contact of the pacing electrode during periods of reperfusion while maintaining blood flow.

3. The system of claim 1, wherein the at least one balloon includes two balloons adapted to assist in delivering cardiac protection therapies.

4. The system of claim 3, wherein the two balloons include:
an outer balloon facing the vessel wall, the outer balloon applying pressure against the vessel wall without filling an inner lumen of the spiral; and
an inner balloon facing a vessel lumen, the inner balloon adapted to fill to occlude blood flow.

5. The system of claim 4, wherein at least one electrode is incorporated onto the outer balloon.

6. The system of claim 1, wherein the catheter and balloon are asymmetrically shaped, and the balloon is adapted to maintain contact between an electrode incorporated onto the balloon and the vessel wall when the balloon is inflated.

7. The system of claim 1, wherein the catheter includes a positive flow balloon catheter that includes one or more blood flow channels that can be opened and closed using multiple lumen balloons.

8. The system of claim 1, wherein delivery of the CPPT and the intermittent ischemia are controlled using a closed-loop system monitoring the hemodynamic parameter.

9. The system of claim 1, wherein the at least one hemodynamic sensor includes a pressure sensor.

10. The system of claim 1, further comprising:
at least one sensing electrode along the length of the catheter.

11. The system of claim 1, wherein the catheter is adapted to deploy a stent.

12. A system for delivering cardiac protection therapies to a heart via a blood vessel, comprising:
a catheter having a balloon along its length, the balloon adapted to be placed in the blood vessel to at least partially occlude the blood vessel;
a guide wire adapted to guide placement of the catheter, the guide wire including at least one hemodynamic sensor;
at least one pacing electrode along the length of one of the catheter and the guide wire; and
a controller connected to the catheter and adapted to sense a hemodynamic parameter being a measure of hemodynamic performance using the hemodynamic sensor, and adapted to control inflation and deflation of the balloon to provide intermittent ischemia, and further adapted to control pulses to the at least one pacing electrode to provide cardiac protection pacing therapy (CPPT).

13. The system of claim 12, wherein the guide wire including the at least one hemodynamic sensor includes a flow sensor.

14. The system of claim 12, wherein the guide wire including the at least one hemodynamic sensor includes a pressure sensor.

15. A method for delivering cardiac protection therapies to a heart, comprising:
providing one or more catheters having a balloon, at least one pacing electrode and at least one hemodynamic sensor;
concurrently delivering cardiac protection pacing therapy (CPPT) and intermittent ischemia therapy using the catheter, the pacing and ischemia adapted to protect the heart from ischemic and reperfusion injuries; and
controlling the delivery of the CPPT and the intermittent ischemia using a closed-loop system monitoring a signal sensed by the at least one hemodynamic sensor.

16. The method of claim 15, wherein concurrently delivering CPPT and intermittent ischemia includes alternately delivering CPPT and intermittent ischemia.

17. The method of claim 15, wherein concurrently delivering CPPT and intermittent ischemia includes simultaneously delivering CPPT and intermittent ischemia.

18. The method of claim 15, wherein concurrently delivering CPPT and intermittent ischemia includes delivering CPPT when the balloon is deflated.

19. The method of claim 15, wherein delivering intermittent ischemia therapy includes inflating and deflating the balloon.

20. The method of claim 19, wherein inflating and deflating the balloon is related to an electrocardiogram.

21. The method of claim 20, wherein inflating and deflating the balloon is done on a periodic basis having a period, wherein the period is based on a number of R-waves in the electrocardiogram.

22. The method of claim 15, wherein providing one or more catheters having a balloon includes providing a spiral balloon adapted to enable vessel wall contact of the pacing electrode during periods of reperfusion while maintaining blood flow.

23. The method of claim 15, wherein providing one or more catheters having a balloon includes providing an asymmetrically shaped balloon that contacts the vessel wall during inflation while still allowing blood to perfuse through the balloon.

24. The method of claim 15, wherein providing one or more catheters having at least one hemodynamic sensor includes providing a flow sensor.

25. The method of claim 15, wherein providing one or more catheters having at least one hemodynamic sensor includes providing a pressure sensor.
